(19) United States
(12) Patent Application Publication
(10) Pub. No.: US 2025/0235647 A1
(43) Pub. Date: Jul. 24, 2025

(54) OROPHARYNGEAL AIRWAY INTEGRATED BITE GUARD DEVICE

(71) Applicant: RIYADH Z. ALLY, SCHENECTADY, NY (US)

(72) Inventor: RIYADH Z. ALLY, SCHENECTADY, NY (US)

(21) Appl. No.: 18/417,127

(22) Filed: Jan. 19, 2024

(51) Int. Cl. A61M 16/04

(52) U.S. Cl. CPC A61M 16/0493; A61M 16/0497; A61M 2205/0216; A61M 2210/0625

(57) ABSTRACT

The present invention discloses an improved oropharyngeal airway device used to maintain optimal airway patency and adequate oxygenation. An important aspect of the device is the pharyngeal body which is configured to isolate the dentition and soft tissue, resulting in a larger anterior, posterior and lateral dimensions of the upper airway for improved airflow. The device is an integrated bite guard that provides a soft channel material configured to enclose the entire bite block body to protect one or more incisor teeth and a gum line from dental damage during masseter muscle clenching downward force. The integrated bite guard offers a defined receiving channel as an anchoring contact site for dentition to help maintain optimal device position by limiting undesirable migration of the oropharyngeal airway device.

### BACKGROUND OF THE INVENTION

In modern medicine, the oropharyngeal airway is used to establish and maintain upper airway patency in any situation including but not limited to the patient's own physical pathology as well as medical, surgical, and anesthesia-related causations. The application use of an oropharyngeal airway includes any etiology or pathology which compromises a patient's airway. For example, during cardiopulmonary resuscitation, preexisting medical conditions or medication-induced issues related to anesthesia both during general anesthesia and monitored anesthesia care inclusive of post-anesthesia care scenarios. The intended areas of use for this invention are settings where medical practitioners commonly monitor ventilation and provide airway management for the airway-compromised or unconscious patient. The places where this can be useful include a pre-hospital arena, medical facilities both outpatient centers and in-hospital environments, and by a wide range of medical practitioners in anesthesia, critical care, and pre-hospital medicine.

Obstructed airflow can compromise ventilation and oxygenation, which can lead to hypoxia, hypercarbia, cardiac arrhythmias, and even death. Generally, the oropharyngeal airway prevents upper airway tissue and the tongue from obstructing the airway, facilitating adequate airflow. It has been established that the oropharyngeal airway pulls the mandible and tongue forward in a prognathic position, thereby preventing them from falling against the pharynx and epiglottis. Pressure pushing the pharynx and epiglottis downward obstructs the tracheal opening and laryngeal inlet into the lungs, thus compromising oxygenation and ventilation. Tongue elevation off of the floor of the mouth produced by the oropharyngeal airway creates a larger oral aperture, thus maintaining anterior-posterior dimension for airflow. Establishing this anterior-posterior dimension can also maintain mandibular lift from retrognathia, a condition in which the lower jaw is set further back than the upper jaw. This mandibular drop can place downward force on the tongue causing upper airway obstruction. In addition, the oropharyngeal airway can also maintain lateral dimensions for airflow by stenting a conduit and preventing upper airway soft tissue from collapsing upon itself, which is useful with edentulous populations.

Currently, the modern oropharyngeal airways have been successful in establishing a patient airway but have a design flaw in maintaining its optimal position throughout the duration of its necessity. Displacement of the oropharyngeal airway can further obstruct a patient's breathing and oxygenation. This design flaw allows the device to migrate out of the ideal position related to soft tissue and tongue muscle contraction as well as soft tissue and tongue volume within a semi-confined space pushing against the oropharyngeal airway device, forcing it outward on the path of least resistance and no discernible anchoring attribute to maintain the intended position. Another design flaw of current oropharyngeal airway devices is in their characteristic bite-block bodies which are typically constructed of hard stiff materials which can cause dental and gum damage.

Therefore, there is a need for a device that significantly limits dislodgment and unwanted movement of the oropharyngeal airway device by offering an integrated anchoring point of contact with a characteristic ridge and receiving channel for the patient's dentition. The intended oropharyngeal airway integrated bite guard device design will prevent the dislodgment for the duration of necessity, facilitating desirable positioning for airflow patency of the device. The integrated receiving channel is intended to be constructed of soft material to provide dental protection from substantial masseter muscle biting pressure.

### SUMMARY OF THE INVENTION

The present invention discloses an oropharyngeal airway integrated bite guard device. In particular, one aspect of the present disclosure is directed to an oropharyngeal airway integrated bite guard device, comprising: (a) a pharyngeal body having a buccal end and a pharyngeal end, the pharyngeal body is sized such that when the pharyngeal body is inserted into a user's mouth until the buccal end is disposed outside of the user's mouth and the pharyngeal end is disposed within the pharynx above the epiglottis inside the user's mouth; (b) a bite block body forming a sturdy section at the buccal end of the pharyngeal body configured to isolate one or more incisor teeth to prevent dentition from occluding an airway during mastication; and (c) a bite guard encloses the entire bite block body to protect one or more incisor teeth and a gum line from dental damage during masseter muscle clenching downward force.

In one embodiment, the device is further comprised of an internal conduit channel, which is a hollow inner tubular void enclosed within the pharyngeal body of the device configured to communicate air passage from the pharyngeal end to the buccal end. In another embodiment, the device further comprises a dental channel of designated depression area on the bite guard, which is an anchoring site of contact where the incisors can be seated for one or more teeth or the gum line. In one embodiment, the device further comprises a plurality of dental ridges forming a U-shaped horizontal channel combined with the dental channel providing a designated depression or a trough area for the teeth. In another embodiment, the device further comprises the U-shaped horizontal channel combining with the dental channel providing the dentition anchoring site to limit movement of the oropharyngeal airway bite guard device within the specified area.

Another aspect of the present disclosure is directed to a bite guard device comprising: a dental channel in a depression area on the bite guard, wherein the dental channel is an anchoring site of contact point where the incisors can be seated for one or more teeth or the gum line; and a plurality of dental ridges forming a U-shaped horizontal channel combined with the dental channel providing a designated depression or a trough area for the incisor teeth. In one embodiment, the U-shaped horizontal channel combining with the dental channel provide dentition an anchoring site to limit the movement of the oropharyngeal airway bite guard device within the specified area. In another embodiment, the bite guard is made of soft material to provide dental protection from substantial masseter muscle.

In one embodiment, the bite guard is made of soft material to provide dental protection from substantial masseter muscle. In another embodiment, the bite guard is made of non-brittle, non-toxic and latex-free medical-grade materials. In one embodiment, the bite guard is made from any one of plastic, synthetic plastic, synthetic rubber, or elastomer material. In one embodiment, the bite guard is further made of a thermoplastic elastomers (TPE) material like Silicone. In one embodiment, the bite guard is made of a material having Shore A Hardness between 15 to 35.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1A exemplarily illustrates a perspective view of the oropharyngeal airway integrated bite guard device.

FIG. 1B exemplarily illustrates a side elevation view of the oropharyngeal airway integrated bite guard device of FIG. 1A.

FIG. 1C exemplarily an anterior view of the oropharyngeal airway integrated bite guard device of FIG. 1A.

FIG. 1D exemplarily illustrates a top view of the oropharyngeal airway integrated bite guard device of FIG. 1A.

FIG. 2 exemplarily illustrates a side elevation view of the oropharyngeal airway integrated bite guard device positioned within a user's mouth.

FIG. 3A exemplarily illustrates a side elevation view of the bite guard device.

FIG. 3B exemplarily illustrates a top view of the bite guard device of FIG. 3A.

FIG. 3C exemplarily illustrates a perspective view of the bite guard device of FIG. 3A.

FIG. 4A exemplarily illustrates a side elevation view of the oropharyngeal airway device.

FIG. 4B exemplarily illustrates side perspective view of the oropharyngeal airway device of FIG. 4A.

### DETAILED DESCRIPTION

The present invention provides a solution to the problem of dislodgment and unwanted movement by providing an improved oropharyngeal airway device. In particular, the present invention proposes an oropharyngeal airway integrated bite guard device that significantly limits dislodgment and unwanted movement of the oropharyngeal airway device by offering an integrated anchoring point of contact with a characteristic ridge and receiving channel for the patient's dentition. The integrated receiving channel is intended to be constructed of soft material to provide dental protection from substantial masseter muscle biting pressure.

The present invention discloses an oropharyngeal airway integrated bite guard device, comprising a pharyngeal body having a buccal end and a pharyngeal end, the pharyngeal body is sized such that when the pharyngeal body is inserted into a user's mouth until the buccal end is disposed outside of the user's mouth and the pharyngeal end is disposed within the pharynx above an epiglottis inside the user's mouth. A bite block body forming a sturdy section at the buccal end of the pharyngeal body is configured to isolate one or more incisor teeth to prevent dentition from occluding an airway during mastication. The device further comprises a bite guard that is configured to enclose the entire bite block body to protect one or more incisor teeth and a gum line from dental damage during masseter muscle clenching downward force. The device may further comprise an internal conduit channel that is a hollow inner tubular void enclosed within the pharyngeal body of the device configured to communicate air passage from the pharyngeal end to the buccal end.

The device may comprise a dental channel which is a depression area mounted on the bite guard as an anchoring site of the contact point where the incisors can be seated for one or more teeth or the gum line. The bite guard device may further comprise a plurality of dental ridges forming a U-shaped horizontal channel combined with the dental channel providing a designated depression or a trough area for the incisor teeth. The bite guard may further comprise a dental channel that is an anchoring site of the contact point where the incisors can be seated for one or more teeth or the gum line.

The bite guard may be integrated into the bite block body of the oropharyngeal airway device forming the U-shaped horizontal channel combining the dental channel and one or more dental ridges. The integrated U-shaped anchoring point of the bite guard can maintain optimal device position to free up any medical practitioner to multi-task without the constant distraction of needing to reassess if the oropharyngeal airway is in the correct position.

Moreover, the bite guard may further comprise the U-shaped horizontal channel combined with the dental channel providing the dentition anchoring site to limit the movement of the oropharyngeal airway bite guard device within a specified area for airway patency by providing a designated depression or a trough area for the incisor teeth.

The dental channel may be configured to be made of a soft material to provide dental protection from substantial masseter muscle biting pressure. The entire bite guard is a continuous apparatus that also runs circumferentially around and overlying the bite block body section made of hard material such as PVC (Polyvinyl Chloride). An internal void within the bite guard is sized specifically to allow the reinforced hard inner bite block body structure to be soft padded, offering dental protection against the sturdy central core.

The bite guard can provide the U-shaped horizontal channel depression which serves as the contact point where the front upper and lower teeth incisors can be seated or received for one or more teeth or gum lines as the defined anchoring site. The U-shaped channel may be formed by the designated dental channel of a depression or trough area positioned between two ridge points. The ridge points provide the dentition an anchor point to limit the inward and outward movement of the oropharyngeal airway within the specified area. The intended design will prevent device dislodgment for the duration of necessity, facilitating desirable positioning for airflow patency.

The integrated bite guard device may be constructed of a compliant substance such as but not limited to a non-brittle, non-toxic and latex-free medical grade material. The preferred compliant material should have sufficient tensile strength to withstand substantial masseter muscle biting pressure without causing occlusion. The ductility of the material should be soft enough to allow for minimal compression compliance but stiff enough to not shear off or cause material breakdown. The material characteristic may be, but not be limited to, between 15 and 35 on the Shore A Hardness scale.

The bite guard device may be constructed from any suitable medical grade compliant materials such as but not limited to plastic, synthetic plastic, synthetic rubber, or elastomer material. The bite guard device may be made of a material such as but not limited to latex-free medical grade Silicone that is a hybrid of synthetic rubber and synthetic plastic polymer with sufficient tensile strength to withstand substantial masseter muscle biting pressure without causing occlusion. For example, the bite guard device can be made of a material such as but not limited to thermoplastic elastomers (TPE), for example silicone. Other alternative materials include Styrene Ethylene Butylene Styrene (SEBS) or Mediprene® A. In one embodiment, the bite guard device is made predominantly of silicone.

The device may be constructed by extrusion or injection mold processing. A two-shot injection molding can offer an improved material bonding cohesion between two different constructed material types within a single machining cycle. In the two-shot injection mold processing method, a molten thermoplastic compound of the preferred hardness material PVC is first injected into the mold which is retained in a specific position so only a pre-determined portion of the mold is filled creating a substrate for a second material to be molded around it. The second material such as a molten thermoplastic compound, either silicone, Styrene Ethylene Butylene Styrene (SEBS), or Mediprene® A, is then injected into the remaining space in the mold to complete the process. The framework of the oropharyngeal airway device may be made of material such as but not limited to PVC material, with a preferable compliance of between 85 and 95 on the Shore A Hardness scale.

A description of embodiments of the present disclosure will now be given with reference to the figures. It is expected that the present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes that come within the meaning and range of equivalency of the claims are to be embraced within their scope.

Before any embodiments of the invention are explained in detail, it is to be understood that the disclosure is not limited in its application to the details of construction nor to the arrangement of components set forth in the following description or illustrated in the drawings. The disclosure is capable of other embodiments and of being practiced or of being carried out in various ways.

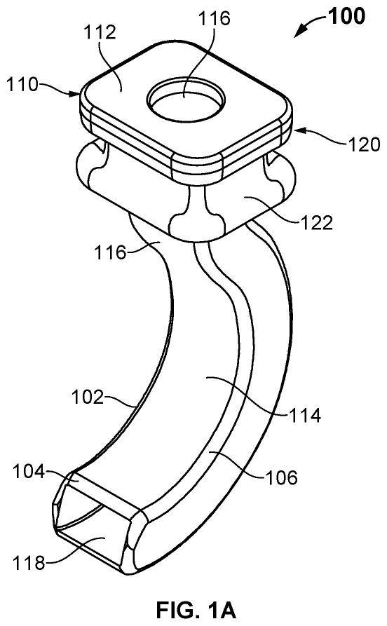

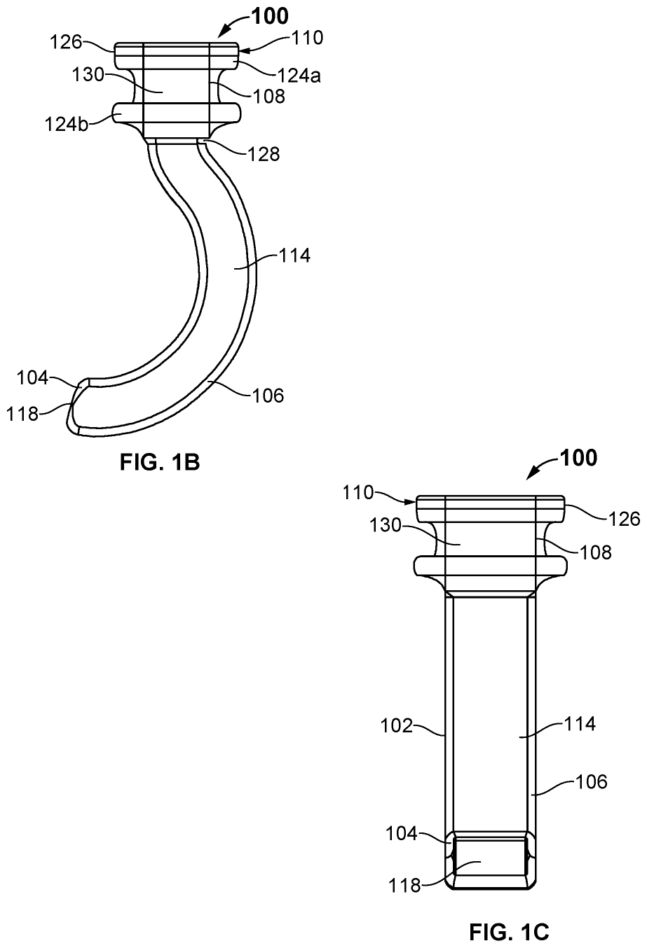

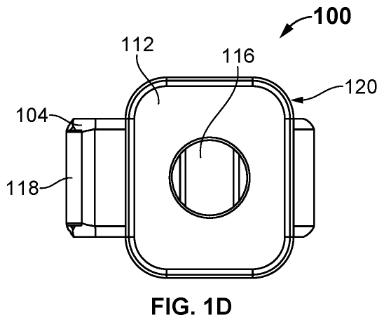

Referring to FIG. 1A to 1D, a schematic diagram of the different views of the oropharyngeal airway integrated bite guard device 100, are illustrated. In one embodiment, the device 100 is configured to deliver quality care for patient populations such as but not limited to compromised patient airway or in an unconscious patient in which the upper airway muscles relax which causes the tongue to push the epiglottis downward thus obstructing the laryngeal inlet restricting access to the lungs. In addition, the relaxed patient's mandible can drop in a retrognathic position that can further place downward force on the soft tissue and the tongue hindering gas exchange. The oropharyngeal airway integrated bite guard device 100 is a combination of integrated bite guard 120 with a basic oropharyngeal airway device 102.

In device 100, the basic oropharyngeal airway device 102 is a continuous body comprised of four main sections: a pharyngeal end 104, a pharyngeal body 106, a reinforced inner bite block body 108 and a buccal end 110. The pharyngeal body 106 has the buccal end 110 and the pharyngeal end 104, in which the pharyngeal body 106 is sized such that when the pharyngeal body 106 is inserted into a user such as the patient's mouth until the buccal end 110 is disposed outside and the pharyngeal end 104 is disposed within the pharynx above an epiglottis. The bite block body 108 forms a receiving channel to engage and isolates one or more incisor teeth to prevent dentition from occluding an airway during mastication.

The buccal end 110 has a flange 112 that sits at the lip line, this flange 112 engages the lips to limit the depth of insertion. The distal area of the buccal end 110 marks the beginning of the reinforced inner bite block body 108 and continues to the beginning of the proximal portion of the pharyngeal body 106. The reinforced bite block body 108 is a sturdy section that engages the teeth and isolates the front upper and lower central or lower lateral incisor teeth to prevent dentition from occluding the airway during mastication. The pharyngeal body 106 begins after the distal portion of the reinforced inner bite block body 108.

The shape of the pharyngeal body 106 has a curved characteristic that conforms to the anatomical shape of the pharyngeal contour and it engages the tongue and functionally lifts the tongue off the posterior oropharynx wall. The pharyngeal end 104 marks the ending of the pharyngeal body 106, which extends into the posterior oropharynx and sits above the epiglottis. The pharyngeal end 104 has a tapered smooth edge to avoid trauma on insertion.

The oropharyngeal airway device 102 further comprises an internal conduit channel 114, a hollow inner tubular void that communicates air passage from the proximal end to the distal end and is completely enclosed within the entire body of the oropharyngeal airway device 102. The internal conduit channel 114 begins at the buccal conduit 116 as a proximal opening on the buccal end 110. It runs continuously as a hollow trapezoidal cross-sectional shape to a distal opening in the pharyngeal conduit area 118 of the pharyngeal end 104. The internal conduit channel 114 allows for unobstructed ventilation and oxygenation that can traverse the defined longitudinal axis pathway.

A framework for the oropharyngeal airway device 102 is made of material such as, but not limited to, PVC material with a preferable compliance of 85 and 95 on the Shore A Hardness scale. The preferred compliant material should have sufficient tensile strength to withstand substantial masseter muscle biting pressure without causing occlusion.

The bite guard 120 is integrated into the inner bite block body 108 of the oropharyngeal airway device 102. The bite guard 120 circumferentially encompasses the reinforced inner bite block body 108 section of the basic oropharyngeal airway device 102 by forging the completed oropharyngeal airway integrated bite guard device 100. The bite guard 120 consists of two main sections such as a dental channel 122 and a two dental ridges (124a and 124b). A U-shaped horizontal channel is formed by the dental channel 122 of a designated depression or trough area positioned between two dental ridge (124a and 124b) points. Combining the dental channel 122 with the two dental ridges (124a and 124b) constructs the U-shape horizontal depression of the contact point where the front upper and lower central or lower lateral incisor teeth or gum line can be received. The designed U-shape horizontal channel characteristic functions as a defined dentition anchoring site to limit the inward and outward movement of the oropharyngeal airway device 102 within a specified area.

The bite guard 120 begins as the proximal bite guard face 126 that is positioned flushed against the lip flange of the buccal end 110. Then continues with the dental ridge 124a that slopes down into the trough area as the dental channel 122 overlying the reinforced inner bite block 108 section. Towards the end of the trough area, the slope rises into the final dental ridge 124b point, upon which it gently slopes downward into the bite guard ending profile 128 as the pharyngeal body 106 curve begins as a continuous contoured trajectory.

The bite guard 120 component is made of a soft compliant continuous material that runs circumferentially overlying the sturdy central core of the basic oropharyngeal airway 102. An internal void 130 permits the bite guard 120 to overlie the reinforced inner bite block body 108, offering dental protection against the sturdy central core.

The device 100 is constructed by an extrusion or injection mold processing. A two-shot injection molding can offer an improved material bonding cohesion between two different constructed material types within a single machining cycle. In the two-shot injection mold processing method, a molten thermoplastic compound of the preferred hardness material PVC is first injected into the mold which is retained in a specific position so only a pre-determined portion of the mold is filled creating a substrate for a second material to be molded around it. The second material such as a molten thermoplastic compound either silicone, Styrene-Ethylene-Butylene-Styrene (SEBS), or Mediprene® A is then injected into the remaining space in the mold to complete the process.

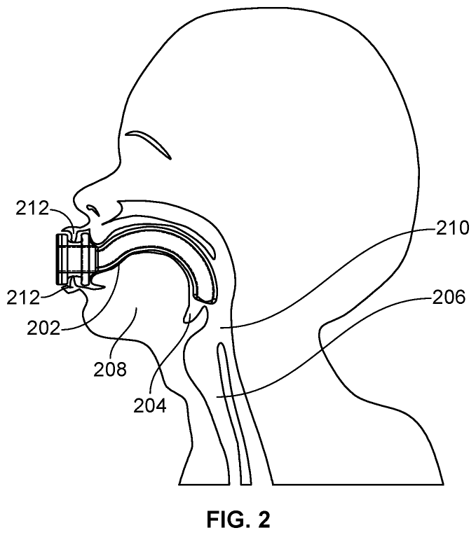

Referring to FIG. 2, a side elevation view of the oropharyngeal airway integrated bite guard device 100 positioned within the user's mouth, is illustrated. FIG. 2 further shows a compromised patient airway in an unconscious patient when the upper airway muscles relax which causes the tongue 202 to push the epiglottis 204 downward thus obstructing the laryngeal inlet 206. This obstruction limits ventilation and oxygen exchange into the laryngeal inlet 206, restricting access to the lungs. In addition, the relaxed patient's mandible 208 can drop in a retrognathic position that can further place downward force on the soft tissue and the tongue 202 hindering gas exchange.

As shown in FIG. 2, the oropharyngeal airway integrated bite guard 100 device is inserted into the patient's mouth extending into their upper airway, keeping the mandible 208 in a prognathic position. This prognathic position prevents the tongue 202 and soft tissue from falling back on the posterior pharyngeal wall 210, which can push the epiglottis 204 downward covering the laryngeal inlet 206.

Further, FIG. 2 illustrates an insertional technique of the oropharyngeal airway integrated bite guard 100 into the patient's mouth, which begins with opening the mouth manually with the practitioner's fingers. Optional use of a tongue blade can be advantageous to depress the tongue 202 caudally, to facilitate a better mouth opening and prevent pushing the tongue 202 backwards during insertion and further obstructing the airway.

The pharyngeal end 104 is pointed toward the corner of the mouth or the mandibular angle, then rotated 90 degrees as the oropharyngeal airway integrated bite guard device 100 is advanced into the posterior oropharynx wall 210 with the pharyngeal end 104 deposited within the posterior oropharynx wall 210 above the epiglottis 204. Once properly inserted, the flange 112 of the buccal end 110 should rest at the lip line. In addition, the front upper and lower central or lower lateral incisor teeth 212 should deposit comfortably within the dental channel 122 and the tongue 202 should rest easily along the bite guard flushed ending profile 128 continuing on the anatomical contour of the pharyngeal body 106.

The pharyngeal end 104 rests between the posterior oropharynx wall 210 and the base of the tongue 202. The positioning and the curved shape of the pharyngeal body 106 will exert pressure along the base of the tongue 202 to push it forward and pull the epiglottis 204 forward with it. This keeps the epiglottis 204 off the posterior pharyngeal wall 210, ensuring airway patency through the laryngeal inlet 206. Appropriately positioned, the oropharyngeal airway integrated bite guard device 100 exerts a forward force on the tongue 202 and the epiglottis 204, this forward force is also transmitted to the mandible 208 pulling it anterior relative to its normal position. The prognathic position of the mandible 208 aids in airway patency and mimics the jaw thrust maneuver. The jaw thrust maneuver is utilized by practitioners to manually displace the mandible 208 forward pulling the tongue 202 forward and preventing it from obstructing airflow into the trachea. A downfall of the jaw thrust maneuver is that it requires the use of at least one hand of the practitioner, which limits multitasking ability. Hence, the oropharyngeal airway integrated bite guard device 100 offers a prognathic hands-free technique.

In device 100, the basic oropharyngeal airway device 102 is a continuous body that consists of four main sections such as the pharyngeal end 104, the pharyngeal body 106, the reinforced inner bite block body 108 and the buccal end 110. The pharyngeal body 106 has the buccal end 110 and the pharyngeal end 104, the pharyngeal body 106 is sized such that when the pharyngeal body 106 is inserted into the user's mouth until the buccal end 110 is disposed outside and the pharyngeal end 104 is disposed within the pharynx above an epiglottis 204. The bite guard 120 forms a receiving channel to engage and isolates one or more incisor teeth 212 to prevent dentition from occluding an airway during mastication.

The buccal end 110 has the flange 112 that sits at the lip line, this flange 112 engages the lips to limit the depth of insertion. The distal area of the buccal end 110 marks the beginning of the reinforced inner bite block body 108 and continues to the beginning of the proximal portion of the pharyngeal body 106. The reinforced bite block body 108 is a sturdy section that engages the teeth and isolates the front upper and lower central or lower lateral incisor teeth to prevent dentition from occluding the airway during mastication. The pharyngeal body 106 begins after the distal portion of the reinforced inner bite block body 108.

The shape of the pharyngeal body 106 has a curved characteristic that conforms to the anatomical shape of the pharyngeal contour and it engages the tongue 202 and functionally lifts the tongue 202 off the posterior oropharynx wall 210. The pharyngeal end 104 marks the ending of the pharyngeal body 106, which extends into the posterior oropharynx wall 210 and sits above the epiglottis 204. The pharyngeal end 104 has a tapered smooth edge to avoid trauma on insertion.

The oropharyngeal airway device 102 is further comprised of an internal conduit channel 114 as a hollow inner tubular void that communicates air passage from the proximal end to the distal end and is completely enclosed within the entire body of the oropharyngeal airway device 102. The internal conduit channel 114 begins at the buccal conduit 116 as a proximal opening on the buccal end 110. It runs continuously as a hollow trapezoidal cross-sectional shape to the distal opening in the pharyngeal conduit area 118 of the pharyngeal end 104. The internal conduit channel 114 allows for unobstructed ventilation and oxygenation that can traverse the defined longitudinal axis pathway.

There are many different techniques to insert the oropharyngeal airway device 102 into the user's mouth, preference of technique is specific to each practitioner but the usage and intended outcome for the oropharyngeal airway integrated bite guard device 100 is universally the same.

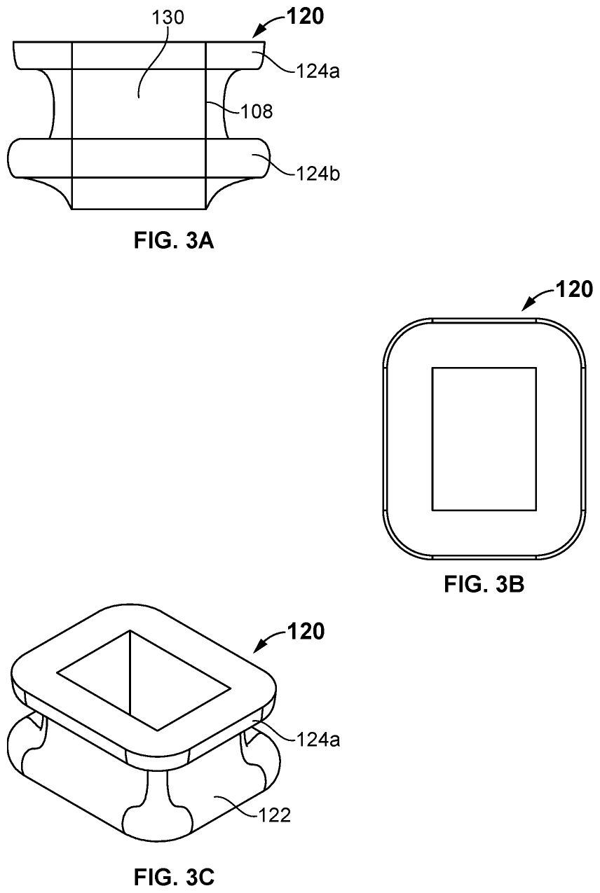

Referring to FIG. 3A to 3C, a side elevation, top and perspective views of the bite guard 120 respectively, are illustrated. The bite guard 120 is integrated into the inner bite block 108 of the oropharyngeal airway device 102. The bite guard 120 circumferentially encompasses the reinforced inner bite block 108 section of the basic oropharyngeal airway device 102 by forging the completed oropharyngeal airway integrated bite guard device 100. The bite guard 120 consists of two main sections such as the dental channel 122 and the two dental ridge (124a and 124b).

A U-shaped horizontal channel is formed by the dental channel 122 of a designated depression or trough area positioned between two dental ridges (124a and 124b) points. Combining the dental channel 122 with the two dental ridges (124a and 124b) constructs the U-shape horizontal depression of the contact point where the front upper and lower central or lower lateral incisor teeth or gum line can be received. The designed U-shape horizontal channel characteristic functions as a defined dentition anchoring site to limit the inward and outward movement of the oropharyngeal airway device 102 within a specified area.

The bite guard 120 begins as the proximal bite guard face 126 that is positioned flushed against the lip flange of the buccal end 110. Then, the proximal bite guard face 126 continues with the dental ridge 124a that slopes down into the trough area as the dental channel 122 overlying the reinforced inner bite block 108 section. Towards the end of the trough area, the slope rises into the final dental ridge 124b point, upon which it gently slopes downward into the bite guard ending profile 128 as the pharyngeal body 106 curve begins as a continuous contoured trajectory.

In the bite guard 120, the dental channel 122 is intended to be made of soft material to provide dental protection from substantial masseter muscle from biting pressure. The entire bite guard 120 is a continuous apparatus that also runs circumferentially around and overlying the inner bite block 108 section made of hard material such as PVC (polyvinyl chloride). The internal void 130 within the bite guard 120 is sized specifically to allow the reinforced hard inner bite block 108 structure to be soft padded, offering dental protection against the sturdy central core.

The integrated bite guard 120 is to be constructed of a compliant substance such as but not limited to a non-brittle, non-toxic and latex-free medical grade material. The preferred compliant material should have sufficient tensile strength to withstand substantial masseter muscle biting pressure without causing occlusion. The ductility of the material should be soft enough to allow for minimal compression compliance but stiff enough to not shear off or cause material breakdown. The material characteristic should be between 15 and 35 on the Shore A Hardness scale.

The bite guard 120 is also constructed from any suitable medical grade compliant materials such as, but not limited to, plastic, synthetic plastic, synthetic rubber, or elastomer material. The preferred compliant material should have sufficient tensile strength to withstand substantial masseter muscle biting pressure without causing occlusion.

In one embodiment, the bite guard 120 is made of a material such as but not limited to latex-free medical grade Silicone as a hybrid of synthetic rubber and synthetic plastic polymer with the sufficient tensile strength to withstand substantial masseter muscle biting pressure without causing occlusion. In yet another embodiment, the bite guard 120 is further made of a material such as but not limited to thermoplastic elastomers (TPE) such as Styrene Ethylene Butylene Styrene (SEBS) or Mediprene® A.

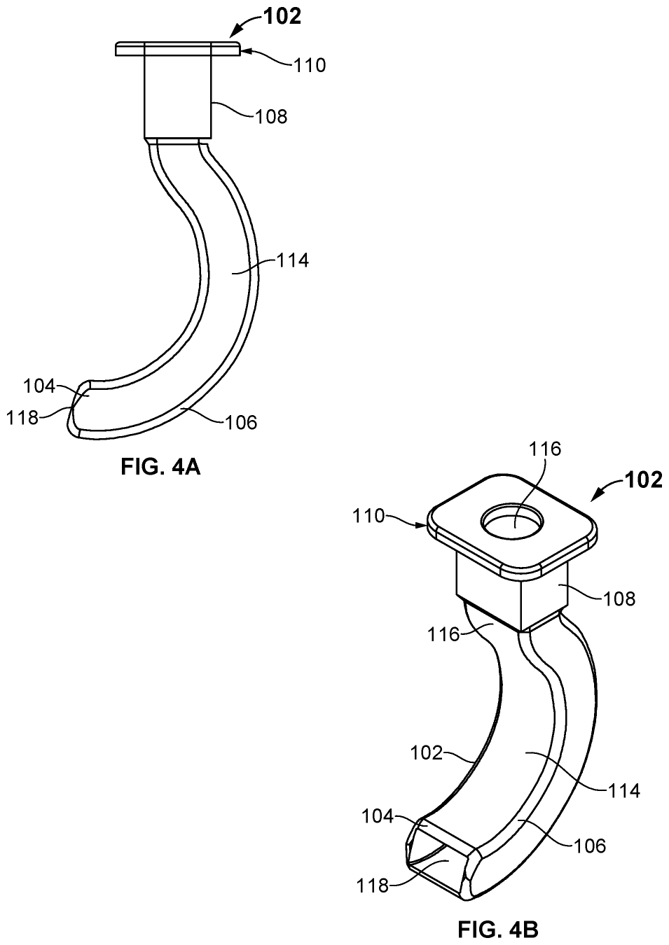

Referring to FIG. 4A and FIG. 4B, a side elevation and side perspective views of the oropharyngeal airway device 102, are illustrated. The oropharyngeal airway device 102 is a continuous body comprises four main sections such as pharyngeal end 104, pharyngeal body 106, reinforced inner bite block body 108 and the buccal end 110. The pharyngeal body 106 has the buccal end 110 and the pharyngeal end 104, the pharyngeal body 106 is sized such that when the pharyngeal body 106 is inserted into the user's mouth until the buccal end 110 is disposed outside and the pharyngeal end 104 is disposed within the pharynx above an epiglottis 204. The bite block body 108 forms a receiving channel to engage and isolates one or more incisor teeth to prevent dentition from occluding an airway during mastication.

The buccal end 110 has the flange 112 that sits at the lip line, this flange 112 engages the lips to limit the depth of insertion. The distal area of the buccal end 110 marks the beginning of the reinforced inner bite block body 108 and continues to the beginning of the proximal portion of the pharyngeal body 106. The reinforced bite block body 108 is a sturdy section that engages the teeth and isolates the front upper and lower central or lower lateral incisor teeth to prevent dentition from occluding the airway during mastication. The pharyngeal body 106 begins after the distal portion of the reinforced inner bite block body 108.

A framework for the oropharyngeal airway device 102 is made of material such as but not limited to PVC material with a preferable compliance of between 85 and 95 on the Shore A Hardness scale. The preferred compliant material should have sufficient tensile strength to withstand substantial masseter muscle biting pressure without causing occlusion.

The oropharyngeal airway integrated bite guard 100 is designed in such a way that significantly limits dislodgment and unwanted movement of the oropharyngeal airway device by offering an integrated anchoring point of contact with a characteristic ridge and receiving channel for the patient's dentition. The integrated receiving channel is intended to be constructed of soft material to provide dental protection from substantial masseter muscle biting pressure.

The proposed device 100 maintains airway patency more effectively by providing the U-shape apparatus that serves as an anchoring point to secure the optimal position. Existing devices do not offer characteristic features to maintain position after the oropharyngeal airway device 102 is established within the user's mouth. In addition, the invention is superior to prior existing devices because it offers a soft compliant material to protect the dentition from damage. The invention consists of the inner hard PVC bite block body 108 which is circumferentially covered with a soft compliant material of bite guard 120. This advantage offers dental protection from the soft material in addition to preventing airway obstruction from biting by maintaining the inner PVC core structure. The invention also provides medical practitioners peace of mind in patient safety by protecting from dental damage. In addition, it also allows practitioners to multi-task without needing to constantly reassess if the oropharyngeal device 102 is maintained in the correct position. The prognathic position of the mandible 208 aids in airway patency and mimics the jaw thrust maneuver where the device 100 does not require manual displacement of the mandible 208 pulling the tongue 202 forward and preventing it from obstructing airflow into the trachea.

The foregoing description comprises illustrative embodiments of the present disclosure. Having thus described exemplary embodiments of the present disclosure, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method.

Many modifications and other embodiments of the disclosure will come to mind to one skilled in the art to which this disclosure pertains having the benefit of the teachings presented in the foregoing descriptions. Although specific terms may be employed herein, they are used only in a generic and descriptive sense and not for purposes of limitation. Accordingly, the present disclosure is not limited to the specific embodiments illustrated herein. While the above is a complete description of the preferred embodiments of the disclosure, various alternatives, modifications, and equivalents may be used. Therefore, the above description and the examples should not be taken as limiting the scope of the disclosure, which is defined by the appended claims.

1. An oropharyngeal airway integrated bite guard device, comprising:
a pharyngeal body having a buccal end and a pharyngeal end, the pharyngeal body is sized such that when the pharyngeal body is inserted into a user's mouth until the buccal end is disposed outside of the user's mouth and the pharyngeal end is disposed within the pharynx above an epiglottis inside the user's mouth;
a bite block body forming a sturdy section at the buccal end of the pharyngeal body configured to isolate one or more incisor teeth to prevent dentition from occluding an airway during mastication; and
a bite guard encloses the entire bite block body to protect one or more incisor teeth and a gum line from dental damage during masseter muscle clenching downward force.

2. The device of claim 1, wherein the device further comprises an internal conduit channel, which is a hollow inner tubular void enclosed within the pharyngeal body of the device configured to communicate air passage from the pharyngeal end to the buccal end.

3. The device of claim 1, wherein the device further comprises a dental channel of designated depression area on the bite guard, which is an anchoring point of contact where the incisors can be seated for one or more teeth or the gum line.

4. The device of claim 1, wherein the device further comprises a plurality of dental ridges forming a U-shaped horizontal channel combined with the dental channel providing a designated depression or a trough area for the teeth.

5. The device of claim 1, wherein the device further comprises the U-shaped horizontal channel combining with the dental channel providing the dentition anchoring site to limit movement of the oropharyngeal airway bite guard device within the specified area.

6. The device of claim 1, wherein the bite guard is made of soft material to provide dental protection from substantial masseter muscle.

7. The device of claim 1, wherein the bite guard is made of non-brittle, non-toxic and latex-free medical-grade materials.

8. The device of claim 1, wherein the bite guard is made from any one of plastic, synthetic plastic, synthetic rubber, or elastomer material.

9. The device of claim 1, wherein the bite guard is further made of a thermoplastic elastomers (TPE) material like Silicone.

10. The device of claim 1, wherein the bite guard is made of a material having Shore A Hardness between 15 to 35.

11. A bite guard device comprising:
a dental channel is a depression area on the bite guard, wherein the dental channel is an anchoring point of contact where the incisors can be seated for one or more teeth or the gum line; and
a plurality of dental ridges forming a U-shaped horizontal channel combined with the dental channel providing a designated depression or a trough area for the incisor's teeth.

12. The device of claim 11, wherein the U-shaped horizontal channel combining with the dental channel provide dentition anchoring site to limit the movement of the oropharyngeal airway bite guard device within the specified area.

13. The device of claim 11, wherein the bite guard is made of soft material to provide dental protection from substantial masseter muscle.

14. The device of claim 11, wherein the bite guard is made of a non-brittle, non-toxic and latex-free medical grade materials.

15. The device of claim 11, wherein the bite guard is made from any one of plastic, synthetic plastic, synthetic rubber, or elastomer material.

16. The device of claim 11, wherein the bite guard is made of a thermoplastic elastomers (TPE) material like Silicone.

17. The device of claim 11, wherein the bite guard is made of a material having a Shore A Hardness between 15 to 35.
